(19) United States
(12) Patent Application Publication
(10) Pub. No.: US 2023/0129533 A1
(43) Pub. Date: Apr. 27, 2023

(54) HYDROGEN SENSITIVE FILM, HYDROGEN SENSOR AND PREPARATION THEREOF

(71) Applicant: YANGTZE DELTA REGION INSTITUTE (QUZHOU), UNIVERSITY OF ELECTRONIC SCIENCE AND TECHNOLOGY OF CHINA, Quzhou (CN)

(72) Inventors: Xiongbang WEI, Quzhou (CN); Jiaxuan LIAO, Quzhou (CN); Wei CHEN, Quzhou (CN); Yuhong QIU, Quzhou (CN); Xin LONG, Quzhou (CN)

(21) Appl. No.: 18/145,904

(22) Filed: Dec. 23, 2022

(30) Foreign Application Priority Data
Dec. 30, 2021 (CN) 202111657471.6

(51) Int. Cl. B01J 23/44; G01N 33/00; B01J 21/06; B01J 35/00; B01J 37/04; B01J 37/00

(52) U.S. Cl. CPC B01J 23/44; G01N 33/005; B01J 21/063; B01J 35/0013; B01J 35/006; B01J 37/04; B01J 37/0036

(57) ABSTRACT

A hydrogen sensitive film, a hydrogen sensor and a preparation thereof. The hydrogen sensitive film has a composite structure of an aerogel and a catalyst. The aerogel can adsorb hydrogen and undergo hydrogenation reaction with hydrogen. The catalyst is a nano-noble metal catalyst for catalyzing the hydrogenation reaction, and is distributed in pores of the aerogel. The hydrogen sensitive film is prepared by mixing a catalyst into an aerogel through physical compounding. The hydrogen sensor includes an insulating substrate layer, the hydrogen sensitive film and an electrode layer.

### CROSS-REFERENCE TO RELATED APPLICATIONS

This application claims the benefit of priority from Chinese Patent Application No. 202111657471.6, filed on Dec. 30, 2021. The content of the aforementioned application, including any intervening amendments thereto, is incorporated herein by reference in its entirety.

### TECHNICAL FIELD

This application relates to hydrogen detection, and more particularly to a hydrogen sensitive film, a hydrogen sensor and a preparation thereof.

### BACKGROUND

As an important reducing gas and clean energy source, hydrogen (H₂) has been extensively applied in chemical, aviation, medical, petrochemical, transportation and energy fields. As a dominant clean fuel, hydrogen is characterized by high energy release per unit volume, and thus has attracted considerable attention in industries of fuel cell and power generation. Unfortunately, hydrogen is extremely flammable and explosive when its concentration in air is higher than a lower flammable limit (4%). Therefore, hydrogen sensors are usually adopted to detect the H₂ leakage.

Selection and preparation of hydrogen sensitive materials play an important role in the fabrication of hydrogen sensors. In terms of the hydrogen sensitive material, the existing hydrogen sensors are mainly classified into metal oxide-based hydrogen sensors, metal oxide semiconductor (MOS) hydrogen sensors, optical fiber hydrogen sensors and palladium (Pd)-based hydrogen sensors. Regarding the existing metal oxide hydrogen sensors, most of them can only detect the H₂ concentration not less than 2%, and thus cannot be used as alarm devices for H₂ leakage.

Therefore, it is urgently needed to develop a material and sensor with great hydrogen sensitivity.

### SUMMARY

Accordingly, this application provides a hydrogen sensitive film and a hydrogen sensor with great hydrogen sensitivity.

Technical solutions of this application are specifically described as follows.

In a first aspect, this application provides a hydrogen sensitive film, wherein the hydrogen sensitive film has a composite structure formed by an aerogel and a catalyst;

the aerogel is configured for hydrogen adsorption and hydrogenation reaction with hydrogen; and

the catalyst is a nano-structured noble metal catalyst for catalyzing the hydrogenation reaction, and is distributed in pores of the aerogel; and the catalyst is attached in the pores of the aerogel in a form of particles.

Regarding the hydrogen sensitive film provided herein, the catalyst is loaded in the pores of the aerogel in the form of particles to form a composite structure. Under the action of the catalyst, hydrogen will participate in the hydrogenation as soon as it is in contact with the aerogel with a large specific surface area, and a sharp decline will occur to the resistance value of the aerogel, facilitating improving the detection sensitivity and shortening the response time.

In addition, the aerogel is a hydrogen sensitive material, which experiences changes in properties (such as resistance) after adsorbing hydrogen, and will restore as the hydrogen is released. In some embodiments, the aerogel has high-density pore distribution and large specific surface area, such as an aerogel with a porous network structure which has a specific surface area greater than 1500 m²/g, a density less than 30 kg/m³ and a porosity more than 99%. Such specific nano-structure contributes to unique performance of the aerogel.

In some embodiments, a pore size of the aerogel is 50-100 nm; and a particle size of the catalyst is 5-20 nm.

In some embodiments, a thickness of the aerogel is 500 nm-5 mm.

In some embodiments, a weight ratio of the aerogel to the catalyst is (50-300):1.

In some embodiments, the aerogel is selected from the group consisting of a titanium dioxide (TiO₂) aerogel, a stannic oxide (SnO₂) aerogel, a cadmium oxide (CdO) aerogel, a cerium dioxide (CeO₂) aerogel, an iron oxide (Fe₂O₃) aerogel, a nickel oxide (NiO) aerogel, a zinc oxide (ZnO) aerogel, an indium(III) oxide (In₂O₃) aerogel and a gallium(III) oxide (Ga₂O₃) aerogel.

In some embodiments, the catalyst is nano-palladium (Pd) catalyst or nano-platinum (Pt) catalyst.

In a second aspect, this application provides a method for preparing the above-mentioned hydrogen sensitive film, comprising:

mixing a catalyst particle into a metal oxide aerogel through physical compounding.

In some embodiments, the method comprises:

mixing the metal oxide aerogel with the catalyst particle followed by grinding to obtain a mixed powder; and

adding deionized water to the mixed powder followed by grinding to obtain a slurry;

wherein a weight ratio of the metal oxide aerogel to the catalyst particle is (50-300):1; and a weight ratio of the deionized water to the mixed powder is (5-15):1.

In a third aspect, this application provides a hydrogen sensor, comprising:

an insulating substrate layer;

a hydrogen sensitive film; and

an electrode layer;

wherein the hydrogen sensitive film is the above-mentioned hydrogen sensitive film; or the hydrogen sensitive film is prepared by the above-mentioned method.

Structurally, the hydrogen sensor comprises a substrate layer, a hydrogen sensitive layer, catalyst particles and an electrode layer, where the hydrogen sensitive layer is an aerogel coating capable of participating in hydrogenation reaction with hydrogen, such as TiO₂. The catalyst particles (such as Pd) are loaded in the aerogel coating to catalyze the hydrogenation reaction. The electrode layer is arranged at a top of the aerogel coating.

The substrate layer is an electric insulating layer, such as an inorganic electric insulator (e.g., glass) and an organic electric insulator (e.g., polyester). The substrate layer can be designed as flat, rod or sphere.

In some embodiments, a thickness of the electrode layer is 20-200 nm.

In a fourth aspect, this application provides a method for preparing the above-mentioned hydrogen sensor, comprising:

(a) mixing the aerogel and the catalyst in water to obtain a slurry;

(b) coating the slurry onto an upper surface of the insulating substrate layer at a desired thickness followed by drying to obtain an aerogel coating; and

(d) preparing the electrode layer on a surface of the aerogel coating to obtain the hydrogen sensor.

The metal oxide aerogel is prepared by supercritical drying or other feasible methods. During the preparation process, the catalyst particles are doped into the metal oxide aerogel through physical compounding to obtain a slurry, which is then applied onto the insulating substrate layer through drop coating, spin coating or blade coating to form the aerogel coating.

Compared to the prior art, this application has the following beneficial effects.

The aerogel is an amorphous solid nanomaterial with low density, large specific surface area and high porosity, which has a three-dimensional (3D) network structure composed of porous nanoparticles. The 3D network structure is filled with air, and the pore size is adjustable within a range from a few nanometers to a few hundred nanometers, as shown in FIG. 6. Based on such special porous network structure, the aerogel has a specific surface area exceeding 1500 m²/g, a density lower than 30 kg/m³, and a porosity greater than 99%. The nanostructure endows the aerogel with unique properties. The TiO₂ aerogel has a large specific surface area and strong adsorption capacity, exhibiting great hydrogen sensitivity. The TiO₂ aerogel undergoes a significant decrease in resistance upon being exposed to hydrogen gas, and the resistance can restore after the gas is released. The TiO₂ aerogel surface has a strong chemisorption effect on dissociated hydrogen atoms, and the charges are partially transferred from H to the conduction band of TiO₂, such that an accumulation layer of electrons is formed on the TiO₂ aerogel surface leading to an increase in the electric conductivity. Upon removing H₁, the transferred electrons return to the H atoms, and the resistance of the TiO₂ aerogel is restored, such that the TiO₂ aerogel has a sensitive resistance response to H₂.

Regarding the hydrogen sensitive film and hydrogen sensor provided herein, an aerogel-noble metal nanoparticle composite structure is used as a hydrogen-sensitive layer, which integrates the excellent hydrogen sensitivity of the metal oxide and the high porosity and large specific surface area of an aerogel material. Moreover, the noble metal nanoparticles (such as Pb) are evenly and deeply dispersed on walls of the pores of the TiO₂ aerogel, such that more hydrogen can be adsorbed on the TiO₂ aerogel surface and oxidized under the catalysis of Pd. After the dissociation, electrons of the hydrogen chemically adsorbed on the TiO₂ aerogel surface are partially transferred to the conduction band, and accumulated on the TiO₂ aerogel surface, allowing for enhanced electrical conductivity. Upon removing the ambient hydrogen, the electrons return to the adsorbed hydrogen, and after the hydrogen is desorbed, the resistance of the TiO₂ aerogel is restored. Due to the high porosity and large specific surface area of the TiO₂ aerogel as well as the high-density distribution of Pd catalyst particles, the hydrogen sensitivity of the hydrogen sensor is greatly improved.

### BRIEF DESCRIPTION OF THE DRAWINGS

The present disclosure will be more apparent and understandable with reference to the accompanying drawings. The accompanying drawings form a part of the disclosure, but are not intended to limit the disclosure.

FIG. 1A is a side view of a plate-shaped hydrogen sensor according to an embodiment of the disclosure;

FIG. 1B is a top view of the plate-shaped hydrogen sensor according to an embodiment of the disclosure;

FIG. 2 is a side view of a rod-shaped hydrogen sensor according to an embodiment of the disclosure;

FIG. 3 schematically depicts an aerogel of the hydrogen sensor according to an embodiment of the disclosure;

FIG. 4 schematically depicts a Pd particle-loaded aerogel of the hydrogen sensor according to an embodiment of the disclosure;

FIG. 5 is a scanning electron microscope (SEM) image of a hydrogen sensor according to Example 1 of the disclosure;

FIG. 6 schematically depicts a structure of the aerogel according to an embodiment of the disclosure;

FIG. 7 illustrates a response of the hydrogen sensor prepared in Example 1 at room temperature under different hydrogen concentrations; and

FIG. 8 illustrates a response of the hydrogen sensor prepared in Embodiment 2 at room temperature under different hydrogen concentrations.

In the drawings: 1, insulating substrate layer; 2, hydrogen sensitive film; and 3, electrode layer.

### DETAILED DESCRIPTION OF EMBODIMENTS

The present disclosure will be described in detail below with reference to the embodiments and accompanying drawings to make objects, technical solutions and advantages of the present disclosure more apparent and understandable. Obviously, described below are merely illustrative, and are not intended to limit the disclosure.

### Example 1

Provided herein was a hydrogen sensor, which was prepared through the following steps.

(S1) Preparation of TiO₂ Aerogel

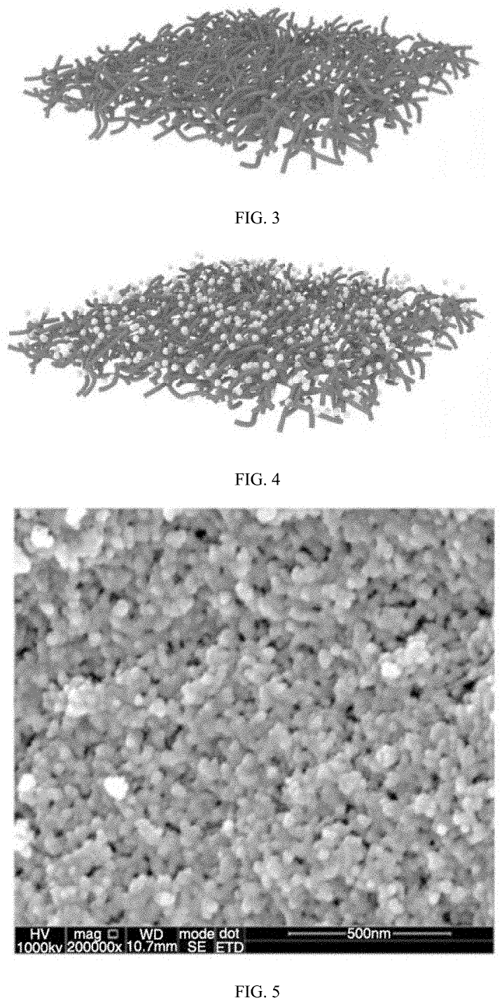

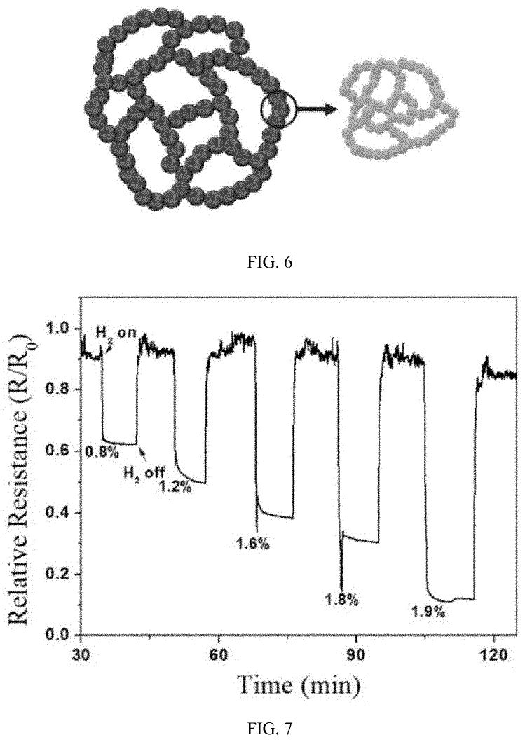

A TiO₂ aerogel with uniform and regular pores was prepared through supercritical drying, where the obtained TiO₂ aerogel had a specific surface area of 1600 m²/g, a density of 35 kg/m³, a porosity of 99%, and a pore size of 50 nm. The prepared TiO₂ aerogel was schematically depicted in FIGS. 3 and 6.

(S2) Preparation of TiO₂ Aerogel-Pd Slurry

After annealed, the TiO₂ aerogel was mixed with Pd powder (particle size: 8 nm) in a mortar in a weight ratio of 60:1 and ground to obtain a mixed powder, which was dropwise added with deionized water and ground evenly to obtain a slurry, where a weight ratio of the deionized water to the mixed powder was 6:1.

(S3) Preparation of Aerogel Coating

The slurry was uniformly coated on a quartz substrate 1, and dried at 80° C. to obtain an aerogel coating. Such process was repeated until a thickness of the aerogel coating 2 reached 500 nm. The microscopic structure of the Pd particle-loaded TiO₂ aerogel was schematically depicted in FIGS. 4-5.

(S4) Preparation of Electrode

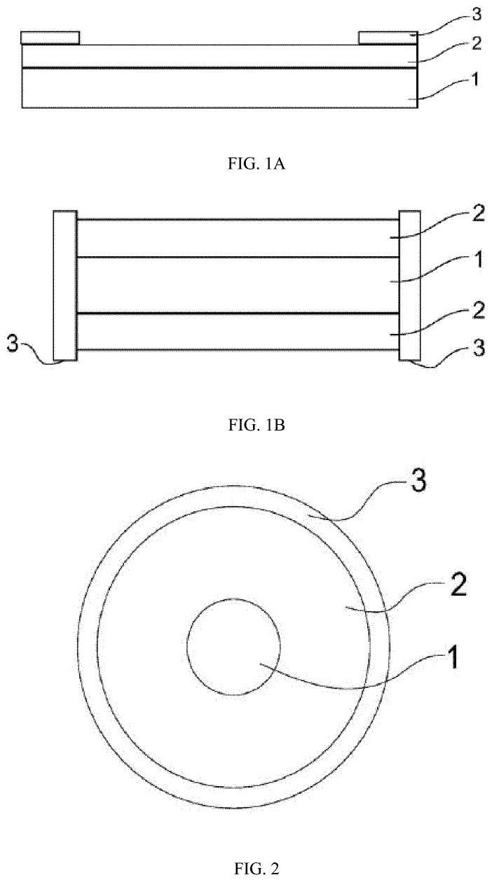

Pt electrodes 3 were deposited on two ends of a surface of the aerogel coating 2 to form effective electrical contact with the aerogel coating, where a thickness of the Pt electrode layer was 30 nm (as shown in FIGS. 1A-B).

The hydrogen sensor prepared herein, which consisted of an insulating substrate layer 1, a hydrogen sensitive film 2 and an electrode layer 3, was tested for the hydrogen sensitivity, and the results demonstrated that the hydrogen sensor exhibited great sensitivity at room temperature. Specifically, the sensitivity of the hydrogen sensor reached 97.8% under a hydrogen content of 1.6 vol %, and the hydrogen sensor had a response time of 2 s and a restoring time of 10 s (as shown in FIG. 7).

### Example 2

Provided herein was a hydrogen sensor, which was prepared through the following steps.

(S1) Preparation of TiO₂ Aerogel

A TiO₂ aerogel with uniform and regular pores was prepared through supercritical drying, where the obtained TiO₂ aerogel had a specific surface area of 1600 m²/g, a density of 35 kg/m³, a porosity of 99%, and a pore size of 50 nm.

(S2) Preparation of TiO₂ Aerogel-Pd Slurry

After annealed, the TiO₂ aerogel was mixed with Pd powder (particle size: 20 nm) in a mortar in a weight ratio of 230:1 and ground to obtain a mixed powder, which was dropwise added with deionized water and ground evenly to obtain a slurry, where a weight ratio of the deionized water to the mixed powder was 12:1.

(S3) Preparation of Aerogel Coating

The slurry was uniformly coated on a surface of a polytetrafluoroethylene (PTFE) rod 1 (length: 5 cm, diameter: 10 mm), and dried at 80° C. to obtain an aerogel coating. Such process was repeated until a thickness of the aerogel coating 2 reached 2 mm.

(S4) Preparation of Electrode

Pt electrodes 3 were deposited on two ends of a surface of the aerogel coating 2 to form effective electrical contact with the aerogel coating. A thickness of the Pt electrode layer was 160 nm (as shown in FIG. 2).

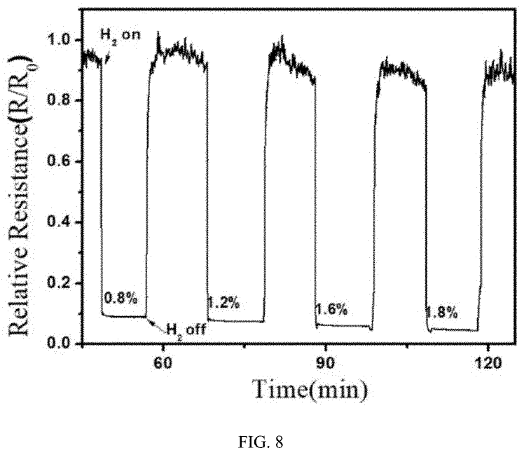

The hydrogen sensor prepared herein was tested for the hydrogen sensitivity, and the results demonstrated that the hydrogen sensor exhibited great sensitivity at room temperature. Specifically, the sensitivity of the hydrogen sensor reached 98.2% under a hydrogen content of 1.6 vol %, and the hydrogen sensor had a response time of 1.7 s and a restoring time of 8 s (as shown in FIG. 8).

Described above are only some embodiments of the present disclosure, which are not intended to limit the disclosure. It should be understood that any variations, replacements and improvements made by those of ordinary skilled in the art without departing from the spirit of the disclosure should fall within the scope of the disclosure defined by the appended claims.

What is claimed is:

1. A hydrogen sensitive film, wherein the hydrogen sensitive film has a composite structure formed by an aerogel and a catalyst;
the aerogel is configured for hydrogen adsorption and hydrogenation reaction with hydrogen; and
the catalyst is a nano-structured noble metal catalyst for catalyzing the hydrogenation reaction, and is distributed in pores of the aerogel.

2. The hydrogen sensitive film of claim 1, wherein a pore size of the aerogel is 50-100 nm; and a particle size of the catalyst is 5-20 nm.

3. The hydrogen sensitive film of claim 1, wherein a thickness of the aerogel is 500 nm-5 mm.

4. The hydrogen sensitive film of claim 1, wherein a weight ratio of the aerogel to the catalyst is (50-300):1.

5. The hydrogen sensitive film of claim 1, wherein the aerogel is selected from the group consisting of a titanium dioxide (TiO₂) aerogel, a stannic oxide (SnO₂) aerogel, a cadmium oxide (CdO) aerogel, a cerium dioxide (CeO₂) aerogel, an iron oxide (Fe₂O₃) aerogel, a nickel oxide (NiO) aerogel, a zinc oxide (ZnO) aerogel, an indium(III) oxide (In₂O₃) aerogel and a gallium(III) oxide (Ga₂O₃) aerogel.

6. The hydrogen sensitive film of claim 1, wherein the catalyst is nano-palladium (Pd) catalyst or nano-platinum (Pt) catalyst.

7. A method for preparing a hydrogen sensitive film, comprising:
mixing a catalyst particle into a metal oxide aerogel through physical compounding.

8. The method of claim 7, wherein the physical compounding is performed through steps of:
mixing the metal oxide aerogel with the catalyst particle followed by grinding to obtain a mixed powder; and
adding deionized water to the mixed powder followed by grinding to obtain a slurry;
wherein a weight ratio of the metal oxide aerogel to the catalyst particle is (50-300):1; and a weight ratio of the deionized water to the mixed powder is (5-15):1.

9. A hydrogen sensor, comprising:
an insulating substrate layer;
the hydrogen sensitive film of claim 1; and
an electrode layer.

10. The hydrogen sensor of claim 9, wherein a thickness of the electrode layer is 20-200 nm.

11. A hydrogen sensor, comprising:
an insulating substrate layer;
a hydrogen sensitive film; and
an electrode layer;
wherein the hydrogen sensitive film is prepared by the method of claim 7.

12. The hydrogen sensor of claim 11, wherein a thickness of the electrode layer is 20-200 nm.

13. A method for preparing a hydrogen sensor, comprising:
(a) mixing an aerogel and a catalyst in water to obtain a slurry;
(b) coating the slurry onto an upper surface of an insulating substrate layer at a desired thickness followed by drying to obtain an aerogel coating; and
(c) preparing an electrode layer on a surface of the aerogel coating to obtain the hydrogen sensor.
